(19) United States
(12) Patent Application Publication
(10) Pub. No.: US 2023/0128592 A1
(43) Pub. Date: Apr. 27, 2023

(54) ULCERATIVE COLITIS DIET, FORMULAE, PRODUCTS AND METHODS THEREOF

(71) Applicant: THE E. WOLFSON MEDICAL CENTER, Holon (IL)

(72) Inventors: Arie LEVINE, Mazkeret Batya (IL); Chen SARBAGILI SHABAT, Rehovot (IL); Rotem SIGALL BONEH, Shimshit (IL)

(21) Appl. No.: 17/915,722

(22) PCT Filed: Mar. 31, 2021
(86) PCT No.: PCT/IL2021/050367

§ 371 Date: Sep. 29, 2022

Related U.S. Application Data
(60) Provisional application No. 63/003,318, filed on Apr. 1, 2020.

(51) Int. Cl. A23L 33/00; A23L 33/115; A23L 33/125; A23L 33/175; A23L 33/19; A23L 33/185; A23L 33/21

(52) U.S. Cl. CPC A23L 33/30; A23L 33/115; A23L 33/125; A23L 33/175; A23L 33/19; A23L 33/185; A23L 33/21

(57) ABSTRACT

An affordable, palatable, maintainable and clinically-useful UC diet, characterized by a low content of sulfur, heme animal-based protein, taurine, animal-saturated fat.

### FIELD OF THE INVENTION

The present invention pertains to an ulcerative colitis diet, formulae, products and methods thereof.

### BACKGROUND OF THE INVENTION

Ulcerative colitis patients are often treated by misleading and out of date nutritional guidelines see e.g., Merchant, Randall E., and Cynthia A. Andre. “A review of recent clinical trials of the nutritional supplement Chlorella pyrenoidosa in the treatment of fibromyalgia, hypertension, and ulcerative colitis.” Alternative therapies in health and medicine 7.3 (2001): 79-92.

Today, this IBD indication is better understood and patient are much effectively treated clinically. Ulcerative colitis is associated with T cell mediated mucosal inflammation with epithelial damage, a damaged or malfunctioning mucous layer, abnormalities and loss of goblet cells. On the microbiome side there appears to be a decrease in specific commensal groups of bacteria that produce butyrate, and conversely an increase in sulfate reducing bacteria and increase in Escherichia and other pathobionts.

Nutritional aspects of UC are also better understood. As for today, UC dietary goals are related, inter alia, to both microbiome and host aspects of the disease, including changes in the gut bacteria involving increased sulfate reducing bacteria and H2S production; increased mucosal bacteria (E. Coli); decreased bacteria that provide butyrate and acetate and injury or dysfunction to protective goblet cells, the mucous layer, colonic epithelium; an increase in T-cell mediated respond; ensuing inflammation may further decrease SCFA signaling and other host related aspects.

UC is usually related or suspected to be related to the following:

UC is associated with altered sulfate reducing bacteria (SRB). SRB are an important source of sulphide production within the lumen. Sulphide levels are increased in IBD patients. Sulphide H2S breaks down the mucus barrier network.

Sulphides have an inhibitory effect on intestinal SCFA metabolism, Pitcher, M. C. L., E. R. Beatty, and J. H. Cummings. “The contribution of sulphate reducing bacteria and 5-aminosalicylic acid to faecal sulphide in patients with ulcerative colitis.” Gut 46.1 (2000): 64-72; Matsuoka, Katsuyoshi, and Takanori Kanai. “The gut microbiota and inflammatory bowel disease.” Seminars in immunopathology. Vol. 37. No. 1. Springer Berlin Heidelberg, 2015 and Ijssennagger, Noortje, Roelof van der Meer, and Saskia W C van Mil. “Sulfide as a mucus barrier-breaker in inflammatory bowel disease?” Trends in molecular medicine 22.3 (2016): 190-199. incorporated herein as references.

Hence, reduction of SRB's, Proteobacteria and Mucolytic Bacteria (Ruminococcus gnavus) are targets for microbiome intervention in UC patients are hence still an unmet goal. Therapeutic measures directed towards the host would include protecting goblet cells and mucous layer; protecting Epithelium; decreasing T-cell activation, increasing T-regs; Butyrate signaling; and decreasing bacterial proximity to epithelium.

Devkota et al., disclose that the sulfite reducer Bilophila wadsworthia blooms in the presence of taurine-conjugated bile acids, leading to inflammation and high colitis scores. It was also founded that Taurine conjugated bile acids depend on the taurine pool. Furthermore, Taurine doesn't have to be conjugated to BA in order to increase sulphide reducing bacteria; see Devkota, Suzanne, et al. “Dietary-fat-induced taurocholic acid promotes pathobiont expansion and colitis in Il10-/- mice.” Nature 487.7405 (2012): 104-108.

Vidal-Lletjós et al. have suggested that high protein diet may affect colonocyte metabolism. It may increase toxic metabolites p-cresol, ammonia (elevates pH, epithelial damage) and H2S and decrease ability to deal with free radicals. This diet may shift SCFA metabolism as protein is used as a carbon source; and it may be a source of sulfated amino acids and taurine; see Vidal-Lletjós, Sandra, et al. “Dietary protein and amino acid supplementation in inflammatory bowel disease course: what impact on the colonic mucosa?” Nutrients 9.3 (2017): 310, A high protein diet may suppress Faecalibacterium prausnitzii see Mu et al. The colonic microbiome and epithelial transcriptome are altered in rats fed a high-protein diet compared with a normal-protein diet. J. Nutr. 2016, 146, 474-483

A few researchers underlined the role of fibers in UC diet, e.g., being a fermentable substrate to bacteria; butyrate is the main fuel of the colonocyte; inhibiting the mucin degradation activity by the microbiota, and saving the protective potential of the mucus barrier; see Desai, Mahesh S., et al. “A dietary fiber-deprived gut microbiota degrades the colonic mucus barrier and enhances pathogen susceptibility.” Cell 167.5 (2016): 1339-1353; M C L Pitcher et al. Gut 2000:

The role of high protein low fiber diet was also discussed in the art; see Anand, Swadha, Harrisharn Kaur, and Sharmila S. Mande, “Comparative in silico analysis of butyrate production pathways in gut commensals and pathogens.” Frontiers in microbiology 7 (2016): 1945.

Regarding tryptophan (Trp), Agus et al., disclosed that the gut microbiota is a crucial actor in human physiology. Many of these effects are mediated by metabolites that are either produced by the microbes or derived from the transformation of environmental or host molecules. Among the array of metabolites at the interface between these microorganisms and the host is the essential aromatic amino acid tryptophan. In the gut, the three major Trp metabolism pathways leading to serotonin (5-hydroxytryptamine), kynurenine (Kyn), and indole derivatives are under the direct or indirect control of the microbiota:

Alvarado recently showed that in mice, expression of IDO1 in the intestinal epithelial promotes secretory cell differentiation and mucus production; levels of IDO1 are positively correlated with secretory cell markers in ilea of healthy individuals and Crohn's disease patients. We propose that IDO1 contributes to intestinal homeostasis; see Alvarado, David M., et al. “Epithelial Indoleamine 2, 3-Dioxyaenase 1 Modulates Aryl Hydrocarbon Receptor and Notch Signaling to Increase Differentiation of Secretory Cells and Alter Mucus-Associated Microbiota.” Gastroenterology 157.4 (2019): 1093-1108.

The role of high saturated fat in UC diet was further disclosed in the art:

Chassaing et al. disclosed that intestinal tract is inhabited by a large and diverse community of microbes collectively referred to as the gut microbiota. While the gut microbiota provides important benefits to its host, especially in metabolism and immune development, disturbance of the microbiota-host relationship is associated with numerous chronic inflammatory diseases, including inflammatory bowel disease and the group of obesity-associated diseases collectively referred to as metabolic syndrome. A primary means by which the intestine is protected from its microbiota is via multi-layered mucus structures that cover the intestinal surface, thereby allowing the vast majority of gut bacteria to be kept at a safe distance from epithelial cells that line the intestine. Thus, agents that disrupt mucus-bacterial interactions might have the potential to promote diseases associated with gut inflammation. Consequently, it has been hypothesized that emulsifiers, detergent-like molecules that are a ubiquitous component of processed foods and that can increase bacterial translocation across epithelia in vitro, might be promoting the increase in inflammatory bowel disease observed since the mid-twentieth century. Here we report that, in mice, relatively low concentrations of two commonly used emulsifiers, namely carboxymtheylcellulose (CMC) and polysorbate-80, induced low-grade inflammation and obesity/metabolic syndrome in wild-type hosts and promoted robust colitis in mice predisposed to this disorder. Emulsifier-induced metabolic syndrome was associated with microbiota encroachment, altered species composition and increased pro-inflammatory potential. Use of germ-free mice and faecal transplants indicated that such changes in microbiota were necessary and sufficient for both low-grade inflammation and metabolic syndrome. These results support the emerging concept that perturbed host-microbiota interactions resulting in low-grade inflammation can promote adiposity and its associated metabolic effects. Moreover, they suggest that the broad use of emulsifying agents might be contributing to an increased societal incidence of obesity/metabolic syndrome and other chronic inflammatory diseases; See Chassaing, Benoit, et al. “Dietary emulsifiers impact the mouse gut microbiota promoting colitis and metabolic syndrome.” Nature 519.7541 (2015): 92-96.

Dietary additives to avoid are e.g., as follows: E-433 Polysorbate -80, obtained in commercially available products, such as Ice Cream, frozen custard, ice milk, fruit sherbet, and non-standardized frozen desserts' E-466 CMC obtained in commercially available products, such as ice cream, dressing, cheese, icing, toppings, gelatinous desserts, infant/baby formula, candy, cottage cheese, cream cheese spread; and E-407 carrageenan obtained in commercially available products, such as yogurt, chocolate, soymilk, ice cream beverages, nutritional shakes, milk products, milk replacements:

The role of Maltodextrins (MDX) in UC diet was also studied in the art. Laudisi et al., underlined that MDX increases endoplasmic reticulum stress in gut epithelial cells with the downstream disclosed that diets enriched in MDX, but not propylene glycol or animal gelatin, exacerbated intestinal inflammation in both models. Their analysis of the mechanisms suggest the effect of MDX showed up-regulation of inositol requiring protein 1β, a sensor of endoplasmic reticulum stress, in goblet cells, and a reduction of mucin-2 expression with no significant change in mucosa-associated microbiota. Stimulation of murine intestinal crypts and HT29-mtheotrexate treated cell line cells with MDX induced inositol requiring protein 1β via a p38 MAP kinase—dependent mechanism. Treatment of mice with TUDCA prevented mucin-2 depletion and attenuated colitis in MDX-fed mice: see Laudisi, Federica, et al. “The food additive maltodextrin promotes endoplasmic reticulum stress-driven mucus depletion and exacerbates intestinal inflammation.” Cellular and molecular gastroenterology and hepatology 7.2 (2019): 457-473.

Furthermore, Nickerson et al, determined that the polysaccharide dietary additive, maltodextrin (MDX), impairs cellular anti-bacterial responses and suppresses intestinal anti-microbial defense mechanisms. In this addendum, we review potential mechanisms for dietary deregulation of intestinal homeostasis, postulate how dietary and genetic risk factors may combine to result in disease pathogenesis, and discuss these ideas in the context of recent findings related to dietary interventions for IBD; see Nickerson Kourtney P., Rachael Chanin, and Christine McDonald. “Deregulation of intestinal anti-microbial defense by the dietary additive, maltodextrin.” Gut microbes 6.1 (2015): 78-83.

A few inventions present dietary means and methods for treating UC, e.g., WO2018052357 “A dietary supplement for alleviating chronic inflammation in colon” discloses a glycerol ester composition comprising glyceryl valerate, for treating dysbiosis in the colon, the dysbiosis being caused by high fat diets, thereby alleviating long term sequel a chronic inflammation comprising ulcerative colitis in the colon of human. US20150157707A1 “Compositions for treating an intestinal inflammatory condition” likewise discloses a composition comprising at least one human Tr1 cell population directed against a food antigen from common human diet.

None of the above present a stand-alone diet especially tailored for UC-patients and their clinical needs. An ulcerative colitis diet, products and method thereof are still a long-felt need.

### SUMMARY OF THE INVENTION

The present invention hence discloses an affordable, palatable maintainable and clinically-useful UC diet (UCD), characterized by a low content of sulfur, heme animal-based protein, taurine, animal-saturated fat, food-stabilizers, maltodextrins and emulsifiers; and high fiber and tryptophan.

The present invention also discloses an affordable, palatable, maintainable and clinically-useful UC diet, characterized by a low content of sulfur, home animal-based protein, taurine, animal-saturated fat, food-stabilizers, maltodextrins and emulsifiers; and high fiber and tryptophan.

The present invention also discloses an UC diet as defined in any of the above, wherein at least a portion of the fiber is apple pectin; and/or at least a portion of the protein is selected from a group consisting of whey protein, soy protein, goat-milk whey protein and a mixture thereof.

The present invention also discloses an UC diet as defined in any of the above, wherein protein consumption is ranging below 1 gr per kg body per day.

The present invention also discloses an UC diet as defined in any of the above, wherein the protein is not from animal origin and/or wherein the protein from animal origin is reduced.

The present invention also discloses an UC diet as defined in any of the above, wherein the protein is not from animal origin except for lean chicken breast and/or eggs.

The present invention also discloses an UC diet as defined in any of the above, wherein the protein is not from animal origin except for lean chicken breast and/or eggs in combination with whey protein

The present invention also discloses a method of providing ulcerative colitis patients an affordable, palatable, maintainable and clinically-useful UC diet, characterized by providing a dietary formula comprising low content of sulfur, heme animal-based protein, taurine, animal-saturated fat, food-stabilizers, maltodextrins and emulsifiers; and high fiber and tryptophan; and administering about 750 ml of the formula per day during the induction phase.

The present invention also discloses a method as defined in any of the above, wherein at least a portion of the fiber is apple pectin; and/or at least a portion of the protein is selected from a group consisting of whey protein, soy protein, goat-milk whey protein and a mixture thereof.

The present invention also discloses a method as defined in any of the above, wherein protein consumption is ranging below 1 gr per kg body per day.

The present invention also discloses a method as defined in any of the above, wherein the protein is not from animal origin, and/or wherein the protein from animal origin is reduced.

The present invention also discloses a method as defined in any of the above, wherein the protein is not from animal origin except for lean chicken breast and/or eggs.

The present invention also discloses a method as defined in any of the above, wherein protein is not from animal origin except for lean chicken breast and/or eggs in combination with whey protein

The present invention also discloses a method of inducting and maintaining of a remission to an ulcerative colitis patient, administering the patient a diet characterized by a low content of sulfur, heme animal-based protein, taurine, animal-saturated fat, food-stabilizers, maltodextrins and emulsifiers; and high fiber and tryptophan.

The present invention also discloses a method as defined in any of the above, wherein at least a portion of the fiber is apple pectin; and/or at least a portion of the protein is selected from a group consisting of whey protein, soy protein, goat-milk whey protein and a mixture thereof.

The present invention also discloses a method as defined in any of the above, wherein protein consumption is ranging below 1 gr per kg body per day.

The present invention also discloses a method as defined in any of the above, wherein the protein is not from animal origin and/or wherein the protein from animal origin is reduced.

The present invention also discloses a method as defined in any of the above, wherein the protein is not from animal origin except for lean chicken breast and/or eggs.

The present invention also discloses a method as defined in any of the above, wherein the protein is not from animal origin except for lean chicken breast and/or eggs in combination with whey protein

The present invention also discloses a method as defined in any of the above, wherein the method further comprising step of formulating the diet in an edible formula.

The present invention also discloses a method as defined in any of the above, wherein the method further comprising step of administrating the formula to a UC patient in a measure of about 750 ml per day during the induction phase.

The present invention also discloses the diet as defined in any of the above, wherein at least a portion or the whole diet is formulated as a shake or puffed dish.

The present invention also discloses the diet as defined in any of the above, wherein at least a portion or the whole diet is formulated as fluid.

The present invention also discloses the diet as defined in any of the above, wherein at least a portion or the whole diet is formulated as fluid comprising edible solids.

The present invention also discloses the diet as defined in any of the above, wherein at least a portion or the whole diet is a ready-to-use medical food, namely a stand-alone food stuff, exclusively provides the patient all dietary and nutritional requirements.

The present invention also discloses the diet as defined in any of the above, wherein at least a portion or the whole diet is not a stand-alone food stuff, namely other foods are also to be consumed on a daily basis.

The present invention also discloses the diet as defined in any of the above, wherein at least a portion or the whole diet is being configured to be served cool or chill.

The present invention also discloses the diet as defined in any of the above, wherein at least a portion or the whole diet is configured to be served at ambient temperature of hot.

The present invention also discloses the diet as defined in any of the above, wherein at least a portion or the whole diet is being configured to be served as food supplement to a predefined UC-diet.

The present invention also discloses a low protein diet to (i) prevent suppression of Faecalibacterium prausnitzii; (ii) prevent increase in sulfur reducing bacteria; and (iii) reduce goblet cell damage; the diet characterized by a low content of sulfur, heme animal-based protein, taurine, animal-saturated fat, food-stabilizers, maltodextrins and emulsifiers, and high fiber, and tryptophan.

The present invention also discloses a low protein diet as defined in any of the above, wherein at least a portion of the fiber is apple pectin; and/or at least a portion of the protein is selected from a group consisting of whey protein, soy protein, goat-milk whey protein and a mixture thereof.

The present invention also discloses a low protein diet as defined in any of the above, wherein protein consumption is ranging below 1 gr per kg body per day.

The present invention also discloses a low protein diet as defined in any of the above, wherein the protein is not from animal origin and/or wherein the protein from animal origin is reduced.

The present invention also discloses a low protein diet as defined in any of the above, wherein the protein is not from animal origin except for lean chicken breast and/or eggs.

The present invention also discloses a low protein diet as defined in any of the above, wherein the protein is not from animal origin except for lean chicken breast and/or eggs in combination with whey protein

The present invention also discloses a method for increasing Faecalibacterium prausnitzii; decreasing sulfur reducing bacteria; and reducing goblet cell damage. The method comprising step or steps of administering a formula characterized by a low protein, low content of sulfur, heme, animal-based protein, taurine, animal-saturated fat, food-stabilizers, maltodextrins and emulsifiers; and high fiber, and tryptophan.

The present invention also discloses a method as defined in any of the above, wherein at least a portion of the fiber is apple pectin; and/or at least a portion of the protein is selected from a group consisting of whey protein, soy protein, goat-milk whey protein and a mixture thereof.

The present invention also discloses a method as defined in any of the above, wherein the step(s) of administering is providing a patient with a measure about 750 ml of the formula per day during the induction phase

The present invention also discloses a method as defined in any of the above, wherein protein consumption is ranging below 1 gr per kg body per day.

The present invention also discloses a method as defined in any of the above, wherein the protein is not from animal origin and/or wherein the protein from animal origin is reduced.

The present invention also discloses a method as defined in any of the above, wherein the protein is not from animal origin except for lean chicken breast and/or eggs.

The present invention also discloses a method as defined in any of the above, wherein the protein is not from animal origin except for lean chicken breast and/or eggs in combination with whey protein.

The present invention also discloses a food composition for providing a user with diet useful for preventing excess sulfur and taurine as well as for generating ammonia and hydrogen sulfide thereby inhibiting mitochondrial respiration and the mucous layer; the food composition comprising a low content of sulfur, heme animal-based protein, taurine, animal-saturated fat, food-stabilizers, maltodextrins and emulsifiers; and high fiber and tryptophan.

The present invention also discloses a food as defined in any of the above, wherein at least a portion of the fiber is apple pectin; and/or at least a portion of the protein is selected from a group consisting of whey protein, soy protein, goat-milk whey protein and a mixture thereof.

The present invention also discloses a food as defined in any of the above, wherein protein consumption is ranging below 1 gr per kg body per day.

The present invention also discloses a food as defined in any of the above, wherein the protein is not from animal origin and/or wherein the protein from animal origin is reduced.

The present invention also discloses a food as defined in any of the above, wherein the protein is not from animal origin except for lean chicken breast and/or eggs.

The present invention also discloses a food as defined in any of the above, wherein the protein is not from animal origin except for lean chicken breast and/or eggs in combination with whey protein

The present invention also discloses a method for providing a user with a diet for preventing excess sulfur and taurine as well as for generating ammonia and hydrogen sulfide thereby inhibiting mitochondrial respiration and mucous layer; the method comprising step or steps of administering a user a low content of sulfur, home animal-based protein, taurine, animal-saturated fat, food-stabilizers, maltodextrins and emulsifiers; and high fiber and tryptophan.

The present invention also discloses a method as defined in any of the above, wherein at least a portion of the fiber is apple pectin; and/or at least a portion of the protein is selected from a group consisting of whey protein, soy protein, goat-milk whey protein and a mixture thereof.

The present invention also discloses a method as defined in any of the above, wherein protein consumption is ranging below 1 gr per kg body per day.

The present invention also discloses a method as defined in any of the above, wherein n the protein is not from animal origin and/or wherein the protein from animal origin is reduced.

The present invention also discloses a method as defined in any of the above, wherein the protein is not from animal origin except for lean chicken breast and/or eggs.

The present invention also discloses a method as defined in any of the above, wherein the protein is not from animal origin except for lean chicken breast and/or eggs in combination with whey protein

The present invention also discloses an edible formula as defined in any of the above, comprising canola oil, ranging from about 50 to about 80% of fat (wt percentage) MCT from about 15 to about 25%.

The present invention also discloses an edible formula as defined in any of the above, wherein amount of apple pectin ranging from about 0.5 to about 2 gram per 100 ml.

The edible formula of claim 27, as defined in Table 1.

The present invention also discloses an affordable, palatable, maintainable and clinically-useful UC diet, characterized low content of at least one ingredient selected from a group consisting of sulfur, heme animal-based protein, taurine, animal-saturated fat, food-stabilizers, maltodextrins and emulsifiers; and by high content of at least one ingredient selected from a group consisting of fiber and tryptophan.

The present invention also discloses the UC diet as defined in any of the above, wherein at least a portion of the fiber is apple pectin; and/or at least a portion of the protein is selected from a group consisting of whey protein, soy protein, goat-milk whey protein and a mixture thereof.

The present invention also discloses the UC diet as defined in any of the above, wherein protein consumption is ranging below about 1 gr per kg body per day.

The present invention also discloses the UC diet as defined in any of the above, wherein the protein is not from animal origin and/or wherein the protein from animal origin is reduced.

The present invention also discloses the UC diet as defined in any of the above, wherein the protein is not from animal origin except for lean chicken breast and/or eggs.

The present invention also discloses the UC diet as defined in any of the above, wherein the protein is not from animal origin except for lean chicken breast and/or eggs in combination with whey protein

The present invention also discloses an edible formula derived from diet as defined in any of the above.

The present invention also discloses an edible formula derived from diet as defined in any of the above, wherein at least one ingredient is a particulate matter.

The present invention also discloses an edible formula derived from diet as defined in any of the above, wherein it further comprising at least one additive and/or at least one nutraceutical.

The present invention also discloses a method for producing an affordable, palatable, maintainable and clinically-useful UC diet and formulae thereof, characterized by step(s) of admixing low content of sulfur, heme animal-based protein, taurine, animal-saturated fat, food-stabilizers, maltodextrins and emulsifiers; and high fiber and tryptophan.

The present invention also discloses the method as defined in any of the above, wherein at least a portion of the fiber is apple pectin; and/or at least a portion of the protein is selected from a group consisting of whey protein, soy protein, goat-milk whey protein and a mixture thereof.

The present invention also discloses the method as defined in any of the above, wherein protein consumption is ranging below about 1 gr per kg body per day.

The present invention also discloses the method as defined in any of the above, wherein the protein is not from animal origin and/or wherein the protein from animal origin is reduced.

The present invention also discloses the method as defined in any of the above, wherein the protein is not from animal origin except for lean chicken breast and/or eggs.

The present invention also discloses the method as defined in any of the above, wherein the protein is not from animal origin except for lean chicken breast and/or eggs in combination with whey protein.

### DETAILED DESCRIPTION OF THE PREFERRED EMBODIMENT

In the following detailed description, reference is made to the accompanying drawings that form a part hereof, and in which is shown by way of illustration, specific embodiments in which the invention may be practiced. These embodiments are described in sufficient detail to enable those skilled in the art to practice the invention, and it is to be understood that other embodiments may be utilized. It is also to be understood that structural, procedural and system changes may be made without departing from the spirit and scope of the present invention. The following detailed description is, therefore, not to be taken in a limiting sense, and the scope of the present invention is defined by the appended claims and their equivalents.

As used herein, the term “about” refers to any value being smaller than or grater than 25% of the defined measure.

The term “low” refers to any value being zero, or otherwise smaller than 0.5% (wy/wt of the defined formula or diet). According to yet another embodiment of the invention, the term low refers to a value or measure being smaller than about 1%. According to yet another embodiment of the invention, the term low refers to a value or measure being smaller than about 2.5%. According to yet another embodiment of the invention, the term low refers to a value or measure being smaller than about 5%. According to yet another embodiment of the invention, the term low refers to a value or measure being smaller than about 7.5%.

The term “high” refers to any value being greater than 10% (wy/wt of the defined formula air diet). According to yet another embodiment of the invention, the term low refers to a value or measure being greater than about 15%. According to yet another embodiment of the invention, the term low refers to a value or measure being greater than about 20%. According to yet another embodiment of the invention, the term low refers to a value or measure being greater than about 25%. According to yet another embodiment of the invention, the term low refers to a value or measure being greater than about 35%.

The terms “ulcerative colitis” and “UC” interchangeably refers to an IBD usually characterized as a long-term condition that results in inflammation and ulcers of the colon and rectum. It is also in the scope of the invention, that according a few embodiments, those two terms also refer to Crohn's disease. It is also in the scope of the invention, that according a few embodiments, those two terms also refer to any inflammatory bowel diseases. It is also in the scope of the invention, that according a few embodiments, those two terms also refer to any autoimmune disorders, including at least one of a group consisting of diabetes mellitus; rheumatoid arthritis (RA); Psoriasis/psoriatic arthritis; Multiple sclerosis (MS); Systemic lupus erythematosus (SLE); Addison's disease; Graves' disease ; Sjögren's syndrome; Hashimoto's thyroiditis; Myasthenia gravis; Autoimmune vasculitis; Pernicious anemia; and celiac disease.

The terms “diet” and “formula” refer to a composition administered into the body of the patient. One method of such an administration is orally, namely per os. Another method of such an administration is Intraosseous infusion, namely iv. Another method of such an administration is by enteral feeding. Other methods of fluid administration are also utilizable.

The terms “diet” and “formula” refer to a composition provided as a fluid (e.g., soup, drink, jelly), semi-solid (e.g., puree), solid (e.g., dried matter) and any combination thereof.

The terms “diet” and “formula” refer to a composition tha may comprise one or more additives, medicaments, and/or nutraceuticals. As used herein, the term “additive” is intended to encompass any type of food ingredient added to the food product at any time during manufacturing. A “topping” is one type of additive which typically stays on “top” or exterior surface of the cereal base (or a half-product pellet), although a “topping” can also be applied as a “coating” such that it adheres to some or all of the cereal base (or half-product pellet), with or without the assistance of a carrier substance. Liquids in any form are also considered to be additives. Embodiments that discuss the use of “toppings” can also include the use of any type of “additive.” Additives can also be considered to include “color agents” as defined below. Additives also include non-nutritive (non-carbohydrate) high-potency sweeteners (such as aspartame, acesulfame K, and saccharin) as well as carbohydrate-based sweeteners, and any other “carbohydrate” as defined below. Additives further include acids (such as fruit-flavor enhancing edible organic acids, such as citric, malic and/or succinic acid), bases, salts, buffering systems, chelating agents, antioxidants, antimicrobial agents, gases/propellants, and so forth. Additives further include nutrient and health additives such as vitamins, minerals, encapsulated biologically active components, nutraceuticals (defined below), dietary supplements, anti-oxidants, fibers, fructo-oligo saccharides such as inulin, calcium materials such as calcium carbonate and calcium phosphate salts, probiotic bacteria sprinkles (e.g., lactobacillus or acidophilus), energy additives, protein powders, powdered milk fractions, protein or satiety additives, herbs, aromatic substances, and other similar health-enhancing additives. Additives can also include “particulates” as defined herein.

The term “ingredient” as used herein, is the smallest, non-divisible part of a cereal or other food product. For example, a corn flake or a nut cluster is an ingredient. Bases, particulates and clusters are all ingredients.

The term “particulates” is generally used to refer to non-grain items. The term as used herein includes, but is not limited to, added particles such as dried whole fruits (e.g., raisins, dates, blueberries, peaches, raspberries, apricots, strawberries, cranberries, tropical [e.g., pineapple, papaya and mango], etc.), fruit parts (e.g., banana chips, apple chunks, etc.), dried fruit products (whether or not infused with sugar, glycerol, etc.), marshmallows, marshmallow bits (dried or moist), malted milk balls, chocolate and peanut butler chunks, chocolate (e.g., milk chocolate, dark chocolate, white chocolate, etc.), chocolate products (e.g., chocolate-coated raisins, chocolate-coated peanuts, etc.), nuts (e.g., walnuts, raisin nuts, pecans, peanuts, almonds, hazel nuts, macadamia nuts, etc.), shredded coconut, yogurt chips (e.g., vanilla, blueberry, strawberry), clusters of particulates (e.g., honey nut clusters), and so forth.

The term “customized food product” as used herein means a food product that contains any type of customized food ingredients, such as a cereal base (or even a half-product pellet that still requires puffing) together with selected additives designed to meet the needs of a particular consumer. This includes a customized food product which can be served hot, warm, frozen, chilled or at room temperature. A customized food product can also be used as a topping, as an additional ingredient which is either admixed or blended into any other food, including but not limited to, a liquid or semi-liquid, which can be frozen, chilled, warm, hot or at room temperature, i.e., at any desired temperature. A customized food product includes any type of snack, e.g., snack bar, snack chip, pretzel, snack mix, power bars, granola mixes, popcorn snacks, etc. A customized food product can also be any type of portable food (e.g., snack bar), as well as a dessert or meal, including any type of baked goods, fried foods, grilled food and cooked food. In addition to cereals and snacks, examples of customized food products include any type of cereal-based or non-cereal based hot or cold beverages, including but not limited to energy beverages, nutraceutical beverages, teas or tea beverages (e.g., chai), blended beverages (e.g., coffee drinks, alcoholic drinks, etc.), juices and juice blends (i.e., juice/dairy, juice/soda, chilled fruit smoothies), grain-based beverages (i.e., soy milks, oat milks, nut milks, e.g., almond milk, etc.), further including grain/dairy, grain/juice, grain/dairy juice blends), dairy based (i.e., yogurt beverages, flavored milks, etc.), fermented beverages (i.e., dairy based, grain-based, e.g., beer, etc., with or without various pre- and pro-biotic components), fermented solids (e.g., breads, cheeses, with or without various pre-and pro-biotic components), yogurts, fruit-blended foods, fruit/nut blends, gelatins, ice creams, sherbets, flavored frozen bars, frozen novelty treats, hot cereals containing finely ground puffed pieces and cereal-based snacks of varying sizes, and so forth.

Customized food products also include any type of mixes, e.g., dessert mixes, bread mixes, and so forth, as well as pasta blends, meal mixes, dinner mixes (e.g., pasta with seasonings for meats and further including meats, such as freeze-dried meats), side dish mixes, and so forth. Customized food products also include any type of fruits or vegetables as well as fruit or vegetable blends, fruit or vegetable/sauce blends, salad blends, e.g., custom blends of greens and vegetables with custom selected dressings and condiments. Customized food products can also include meats, poultry, beans, pasta, sauces, i.e., virtually any type of food product to which customized food ingredients can be added or which can be created from customized blends of ingredients. This further includes customized food products in which any type of additive has been applied as a coaling, topping, glazing, an additional ingredient, and so forth. A customized food product can also refer to any type of customized animal food, such as for pets, livestock, and so forth.

The term “nutraceutical” as used herein refers to edible materials having, or believed to have, medicinal or even therapeutic effects. Nutraceuticals include the tocopherols, B vitamins, ginseng and other herbs, wheat grass and barley grass and extracts of the grasses, soy-based estrogen analogs or soy isoflavones, chromium picolinate, red rice yeast, minerals, St. John's wort, chitosan, and so on. The term also referring, according t a few embodiments of the invention, to curcumin, cannabinoids (including CBD, THC etc.), probiotics etc.

The term “flavor,” “flavor agent” or “flavoring” as used herein refers to an organoleptic agent in the form of an emulsion, concentrate, aqueous- or oil-soluble liquid or a dry powder, as well as any type of chunky piece or pieces that may be added to a mixture at any time in the process or mixtures thereof, such as a liquid and powder slurry. Flavorings can also be considered additives and can include nuts, nut pieces, fresh fruits, dried fruits, fruit products, candies, marshmallows, dried marshmallow pieces known as “marbits,” chocolates and chocolate products, and so forth. Flavorings further include any fruit flavors such as berry flavors, apple, cherry, plum, raisin, banana, pear, peach, figs, dates and so on. Flavorings may also include fats, salts, honeys, cheeses, frosting, powdered food products, sugar, sugar substitutes, gelatins and spices. Flavorings may also include colorings as well as any nut flavors as well as any sweet flavors such as chocolate, vanilla, caramel, butterscotch, lemon, malt, cinnamon, graham, coconut flavors, mint and so on. Flavorings additionally include any savory flavors such as all meat, game, fowl, fish, dairy, barbecue, smoke, pepper, spicy and vegetable flavors.

The term “sugar” as used herein refers to substantially all sugars and sugar substitutes, including any monosaccharide such as glucose or fructose, disaccharides such as lactose, sucrose or maltose, polysaccharides such as starch, oligosaccharide, sugar alcohols, or other carbohydrate forms such as gums that are starch based, vegetable based or seaweed based (beta glucan, psyllium).

The term “sweetener” as used herein refers to essentially all sweeteners that are “carbohydrate”-based, as defined above under “carbohydrate” and further includes sweeteners that are “non-nutritive” as defined above under “additive.”

The term “fat” as used herein is synonymous with the term “lipid” and refers to substantially all fats and fat mimics (e.g., sucrose polyesters), including any animal (e.g., dairy, marine, etc.) or vegetable fat in solid or liquid form.

The term “color” or “coloring agent” as used herein refers to natural or uncertified colors from natural sources or certified colors for the effect of color. In one embodiment, the colors include dyes, certified aluminum lakes or colors derived from a natural source. Coloring agents may also be water-based or oil-based or dry. Coloring agents may be primary colors, blends of colors or discrete mixtures of colors, such as confetti.

The term “carbohydrate” refers to specific definitions e.g., as in Example 1, yet also, in a some extant, to any organic compound (and its derivatives and analogs) containing carbon, hydrogen, and oxygen as well as a saccharose group, as is known in the art. As such, carbohydrates include mono-, di-, oligo-, and polysaccharides and their derivatives (such as sugar alcohols and sugar esters). Carbohydrates may impart sweetness (as in the case of sugar) or non-sweetness (as in the case of starch). Examples of sweet and non-sweet carbohydrates include fructose, sucrose, lactose, maltose, galactose, xylose, dextrose, maltose, trehalose, raffinose, stachyose, corn syrups, honey, molasses, malt syrups, corn syrup solids, maltodextrins, starches, pectin, gums, carrageenan, and inulin.

The term “puffed” is used herein to collectively refer to a variety of finished forms, including, but not limited to, puffs, flakes, shreds, finely ground particles and so forth. “Puffed” pieces generally refer to cereal pieces having a specific density typically ranging from about 0.15 to 0.3 g/cc. Quantities of pieces of such puffed pieces will have even lower bulk densities (e.g., ten (10) oz. per 200 cu in.). Such puffed pieces are distinguished from “un-puffed” or “half-product” pieces having little or no degree of expansion and generally characterized by specific densities of about 0.3 to 0.8 g/cc. Pieces can be two dimensional of either regular shape (round, oval, square, rectangular) or irregular shape (flake, having a periphery forming an outline of a figurine). Such pieces can have opposed major sides and a thickness of less than about (4) mm, which may be flat, curved or curvilinear, or three-dimensional (3D) (i.e., haying an aspect ratio of any two dimensions ranging from 1:1 to 10:1). 3D shapes can be simple (e.g., a disk or a sphere) or complex such as an airplane or figurine, or spiral.

As used herein, the term “puffed” or “expanding” refers to a drying process in which the half-product is dried rapidly enough to cause the half-product to expand or puff. Puffing occurs when bound moisture in a liquid state is converted to a vapor phase and “suddenly” released during exposure to a suitable energy source, such as thermal or microwave energy. If the half-product is dried too slowly, it remains hard, rather than softening and puffing. This is in contrast to “popping” or “exploding” which occurs when popcorn is popped. This is also in contrast to “cooking,” which is defined herein as the first heat or mechanical treatment that a mixture receives which essentially forms it into a dough. It is this dough, i.e., a processed grain-based unexpanded food product, which is then fabricated into the various half-products described above. (Some of these fabrication processes, such as the forming and flaking mill, as well as other processes further downstream, such as gun puffing, and so forth, can cause the starch in the dough to become gelatinized. As a result, many in the industry refer to the pre-formed dough as only “partially” cooked). It should be noted that the process of “puffing” as described herein, does not cause the starch to become gelatinized. “Puffing” is further distinguished from “reheating” of a finished puffed RTE cereal or snack piece that has a lower moisture content and density, such as about one (1) to about five (5) % moisture and about 0.02 to about 0.7 g/cc, respectively. Such reheating of a finished product would likely be unsuccessful in that it may bum, scorch and/or char the pieces, not generate the fresh flavor, toughen the texture rather than soften the piece, and so forth. “Puffing” is also distinguishable from the gradual expansion of a food product due to an uptake of liquid in the absence of applied energy, which is not a “cooking” process, per se. This includes, but is not limited to, the “soaking” up of a liquid, such as milk, by a conventional cereal product, and so forth. It is important to note, however, that the food products of the present invention can be puffed in the presence of a liquid, such as oil, if desired.

An open label non randomized pilot study was completed. Number of patients recruited 22 UC USA and Israeli patients. Study population is as follows: mild to moderate active UC patients PUCAI 10-45, aged 8-19 years. Duration of the study is 12 weeks.

Inclusion criteria are all of the followings: Informed consent; Established diagnosis of UC disease; Age: 8-19 years; Mild to moderate active disease −10≤PUCAI≥45; and Stable medication (IMM/5ASA) use for the past 6 weeks. Excluding criteria are one of the following: Any proven infection such as positive stool culture, parasite or C. difficile; Use of Antibiotics or steroids in the past 2 weeks; Use of biologics in present or in the past; Acute severe UC (PUCAI>60) in the previous 12 months; Current Extra intestinal manifestation of UC; PSC or Liver disease and Pregnancy.

Pediatric UC diet pilot trial (N=22); The diet induced 73% response, 40% remission at week 6 among 5ASA failures:

Preliminary data from an ongoing trial in adults with the UCD refractory to a medication has disclosed 40% clinical remission at week 8. Adult UCD results in fifteen patients was 40% remission at 8 weeks.

A Formula to be used as a supplement to the diet for treatment of Ulcerative Colitis is prepared, optimized and characterized as follows: the liquid formula is used in conjunction with a diet; the formula is to abide by the principles of the UC Diet previously described; a measure of 750 ml per day (3 glasses) are consumed by patients daily during the induction phase; and the formula is used for induction and maintenance of remission.

Regarding the protein portion of the diet, low -protein formula or “shake” may be provided useful. High protein diets may suppress Faecalibacterium prausnitzii, may provide excess sulfur and taurine as well as generating ammonia and hydrogen sulfide which inhibit mitochondrial respiration. The role of the protein is to provide protein that would not be harmful. The protein source would preferably be e.g., whey protein but soy protein could also be employed. Goat whey was shown to be beneficial in a mouse model of colitis and improve mucin secretion; see Liu, X.; Blouin, J.-M.; Santacruz, A.; Lan, A.; Andriamihaja, M.; Wilkanowicz, S.; Benetti, P.-H; Tomé, D.; Sanz. Y.; Blachier, F.; et al. High-protein diet modifies colonic microbiota and luminal environment but not colonocyte metabolism in the rat model: The increased luminal bulk connection. Am. J. Physiol. Gastrointest. Liver Phvsiol. 2014, 307, G459-G470; Mu, C.; Yang, Y.; Luo, Z.; Guan, L.; Zhu, W. The colonic microbiome and epithelial transcriptome are altered in rats fed a high-protein diet compared with a normal-protein diet. J. Nutr. 2016, 146, 474-483; Vidal Lletjos et al Nutrients 2017, 9, 310; and Araujo D et al. PLOS one 2017 Sep. 28;12(9):e0185382. , all are incorporated herein as a reference.

Regarding the fat portion of the diet, a low animal fat diet with high MUFA low PUFA and saturated fat is preferred. Diet should contain medium-chain triglycerides (MCT) which would decrease primary bile acid secretion. Use of canola oil with MCT is preferable.

Regarding the carbohydrate portion of the diet, well absorbed carbohydrates are unlikely to reach the colon in large amounts. The role of the carbohydrates is to “do no harm” and provide calories. A mix of maltodextrin and sucrose is thus being preferable. Other sources of carbohydrates for the microbiome are providable by diet. Availability of carbohydrate significantly increases palatability of the food and increase its organoleptic properties.

Regarding the fiber portion of the diet, pectin is a family of soluble fibers that we believe are beneficial. Apple pectin is a direct substrate for F. prausnitzii and Eubacterium which are butyrate producers that reduce inflammation. Pectins may stimulate intestinal recovery from colitis via colonic stem cells. It is further noted that as disclosed by Lopez-Siles, Faecalibacterium prausnitzii is one of the most abundant commensal bacteria in the healthy human large intestine, but information on genetic diversity and substrate utilization is limited. Here, we examine the phylogeny, phenotypic characteristics, and influence of gut environmental factors on growth of F. prausnitzii strains isolated from healthy subjects. Phylogenetic analysis based on the 16S rRNA sequences indicated that the cultured strains were representative of F. prausnitzii sequences detected by direct analysis of fecal DNA and separated the available isolates into two phylogroups; See Lopez-Siles, Mireia, et al. “Cultured representatives of two major phylogroups of human colonic Faecalibacterium prausnitzii can utilize pectin, uronic acids, and host-derived substrates for growth.” Appl. Environ. Microbiol. 78.2 (2012): 420-428 Singh, Vishal, et al. demonstrated that pectin had anti-inflammatory properties while inulin was proinflammatory. “Microbiota fermentation-NILRP3 axis shapes the impact of dietary fibres on intestinal inflammation.” Gut 68.10 (2019): 1801-1812, both are incorporated herein as a reference:

An ulcerative colitis formula (UCD) comprising canola oil, ranging from 50-80% of fat (wt)

TABLE 1
An ulcerative colitis diet according
to an embodiment of the invention.

| | | Quan- | | Quan- | |
|---|---|---|---|---|---|
| | | tity in | | tity in | |
| Component | Source | 100 ml | Units | 1000 ml | Units |
| Calories | | 100 | Kcal | 1000 | kcal |
| Protein | Whey protein | 2 | g | 20 | g |
| Carbohydrate | Sucrose and | 11 | g | 110 | g |
| | maltodextrin | | | | |
| Fat total | Canola75%-85% | 4.5 | g | 45 | g |
| | and MCT 15%-25% | | | | |
| Fiber | Apple pectin | 1 | g | 10 | g |

MCT may range from 15-25%. Amount of apple pectin ranging from 0.5 -2 gram per 100 ml.

In an embodiment, the consumer may choose not to complete the survey, but can customize known products with desired additives or ingredients, including vitamins, minerals, herbs, flavors, nutraceuticals, particulates such as fruits and nuts, and so forth. In one embodiment, consumers are invited to select from two to three food products that most closely match their particular needs. In another embodiment, four to one thousand or more choices are presented.

The resulting product can be shipped through various channels of trade in consumer-sized packages to an intermediary or directly to the end user. Packages can include a conventional consumer-sized box, individual pouches, covered bowls or even beverage-type containers. The resulting product is precisely suited to meet the needs of a specific consumer, particularly in the areas of health, taste and texture.

While the invention has been described in detail and with reference to specific embodiments thereof, it will be apparent to on skilled in the art that various changes and modifications can be made therein without departing from the spirit and scope thereof.

1.-72. (canceled)

73. An ulcerative colitis (UC) dietary composition comprising
(a) low content of at least one ingredient selected from sulfur, heme animal-based protein, taurine, animal-saturated fat, food-stabilizers, maltodextrins and/or emulsifiers; and
(b) high content of at least one ingredient selected from fiber and/or tryptophan.

74. The UC dietary composition of claim 73, wherein
(a) low content is smaller than 2.5% (wt/wt) of at least one ingredient selected from sulfur, heme animal-based protein, taurine, animal-saturated fat, food-stabilizers, maltodextrins and/or emulsifiers; and
(b) high content is greater than 10% (wt/wt) of at least one ingredient selected from fiber and/or tryptophan.

75. The UC dietary composition of claim 73 comprising a low content of sulfur, heme animal-based protein, taurine, animal-saturated fat, food-stabilizers, maltodextrins and emulsifiers; and high fiber and tryptophan.

76. The UC dietary composition of claim 75, wherein at least a portion of said fiber is apple pectin; and/or at least a portion of said protein is selected from a group consisting of whey protein, soy protein, goat-milk whey protein and a mixture thereof.

77. The UC dietary composition of claim 75, wherein protein consumption is ranging below 1 gr per kg body per day.

78. The UC dietary composition of claim 75, wherein said protein
(a) is not from animal origin and/or wherein the protein from animal origin is reduced;
(b) is not from animal origin except for lean chicken breast and/or eggs; or
(c) is not from animal origin except for lean chicken breast and/or eggs in combination with whey protein.

79. The dietary composition of claim 73, wherein said formula
(a) is a ready-to-use medical food, namely a stand-alone food stuff, exclusively provides said patient all dietary and nutritional requirements;
(b) is not a stand-alone food stuff, namely other foods are also to be consumed on a daily basis; or
(c) is configured to be served as food supplement to a predefined UC-diet.

80. A method of
(a) inducting and maintaining of a remission to an ulcerative colitis patient in need thereof, said method comprising; or
(b) preventing excess sulfur and taurine and for generating ammonia and hydrogen sulfide thereby inhibiting mitochondrial respiration and mucous layer in a patient in need thereof;
said method comprising administering to said patient a diet comprising low content of sulfur, heme animal-based protein, taurine, animal-saturated fat, food-stabilizers, maltodextrins and emulsifiers; and high fiber and tryptophan.

81. The method of claim 79, wherein at least a portion of said fiber is apple pectin; and/or at least a portion of said protein is selected from a group consisting of whey protein, soy protein, goat-milk whey protein and a mixture thereof.

82. The method of claim 79, wherein protein consumption is ranging below 1 gr per kg body per day.

83. The method of claim 79, wherein said protein
(a) is not from animal origin and/or wherein the protein from animal origin is reduced;
(b) is not from animal origin except for lean chicken breast and/or eggs; or
(c) is not from animal origin except for lean chicken breast and/or eggs in combination with whey protein.

84. The method of claim 79, wherein said diet is administered to said UC patient in a measure of about 750 ml per day during the induction phase.

85. A method of increasing Faecalibacterium prausnitzii; decreasing sulfur reducing bacteria; and reducing goblet cell damage in a patient in need thereof; said method comprising step or steps of administering a formula comprising low protein, low content of sulfur, heme, animal-based protein, taurine, animal-saturated fat, food-stabilizers, maltodextrins and emulsifiers; and high fiber, and tryptophan.

86. The method of claim 85, wherein at least a portion of said fiber is apple pectin; and/or at least a portion of said protein is selected from a group consisting of whey protein, soy protein, goat-milk whey protein and a mixture thereof.

87. The method of claim 85, wherein said step(s) of administering is providing a patient with a measure about 750 ml of said formula per day during an induction phase.

88. The method of claim 85, wherein protein consumption is ranging below 1 gr per kg body per day.

89. The method of claim 85, wherein said protein
(a) is not from animal origin and/or wherein the protein from animal origin is reduced;
(b) is not from animal origin except for lean chicken breast and/or eggs; or
(c) is not from animal origin except for lean chicken breast and/or eggs in combination with whey protein.

90. The UC dietary composition of claim of claim 73, comprising canola oil, ranging from about 50 to about 80% of fat (wt percentage) MCT from about 15 to about 25%; wherein the composition is edible.

91. The UC dietary composition of claim 73, comprising apple pectin; wherein the amount of apple pectin ranges from about 0.5 to about 2 gram per 100 ml; and wherein the composition is edible.

92. The UC dietary composition of claim 73, comprising 2 gram whey protein, 11 gram sucrose and maltodextrin, 4.5 gram fat and 1 gram apple pectin, per 100 ml composition; wherein the composition is edible.
